(19) United States
(12) Patent Application Publication
(10) Pub. No.: US 2022/0133835 A1
(43) Pub. Date: May 5, 2022

(54) ANTIDIABETIC COMPOSITION COMPRISING GINGER EXTRACT OBTAINED FROM MICROWAVE-PROCESSED GINGER AND METHOD OF PREVENTING OR TREATING DIABETIS MELLITUS

(71) Applicant: KOREA INSTITUTE OF SCIENCE AND TECHNOLOGY, Seoul (KR)

(72) Inventors: Jungyeob HAM, Gangneung-si (KR); Taejung KIM, Gangneung-si (KR); Young Tae PARK, Gangneung-si (KR); Pilju CHOI, Gangneung-si (KR); Ji-yool KIM, Gangneung-si (KR); Cheol Hee YOON, Gangneung-si (KR); Haneul JU, Gangneung-si (KR)

(73) Assignee: KOREA INSTITUTE OF SCIENCE AND TECHNOLOGY, Seoul (KR)

(21) Appl. No.: 17/518,799

(22) Filed: Nov. 4, 2021

(30) Foreign Application Priority Data
Nov. 5, 2020 (KR) 10-2020-0147083

(51) Int. Cl. A61K 36/906; A61P 3/10

(52) U.S. Cl. CPC A61K 36/906; A61P 3/10; A61K 2236/39; A61K 2236/333; A61K 2236/10

(57) ABSTRACT

Provided are a pharmaceutical composition for preventing or treating diabetes and a method of preventing or treating diabetes in a subject by using the pharmaceutical composition, the pharmaceutical composition including a ginger extract, as an active ingredient, obtained by microwave irradiation, under pressure, of ginger, an extract thereof, or a combination thereof.

### CROSS-REFERENCE TO RELATED APPLICATION

This application is based on and claims priority under 35 U.S.C. § 119 to Korean Patent Application No. 10-2020-0147083, filed on Nov. 5, 2020, in the Korean Intellectual Property Office, the disclosure of which is incorporated by reference herein in its entirety.

### BACKGROUND

### 1. Field

One or more embodiments relate to an antidiabetic composition and a method of preventing or treating diabetes by using the antidiabetic composition in a subject.

### 2. Description of the Related Art

Diabetes is a type of systemic metabolic disease caused by genetic or environmental factors. Diabetes is mainly classified into insulin-dependent type 1 diabetes and non-insulin-dependent type 2 diabetes. With insulin-dependent type 1 diabetes, symptoms may appear suddenly due to severe insulin deficiency, and critical symptoms such as diabetic ketoacidosis are found. Because type 1 diabetes mainly occurs in young people at the age of 10s to 20s, type 1 diabetes is also known as juvenile diabetes. Type 2 diabetes occurs mainly after the age of 40, and type 2 diabetes patients account for most diabetes patients in Korea. Unlike type 1 diabetes, type 2 diabetes is known as adult-onset diabetes. Although the causes of type 2 diabetes are not clearly identified, it is known that genetic factors and environmental factors are involved in causing type 2 diabetes. Genetic factors show various familial concentrations. For example, identical twins show nearly 90% to 100% disease concordance with type 2 diabetes. It is known that 58% of children suffer from diabetes when both parents suffer from diabetes, and 27% of children suffer from diabetes when one parent suffers from diabetes, and 0.9% of children suffer from diabetes when neither parent suffers from diabetes. Many environmental factors are involved in causing type 2 diabetes, such as high calorie intake, lack of exercise, obesity, stress, and drug abuse. In particular, as the cause of type 2 diabetes, impaired insulin secretion in pancreatic beta cells and defective insulin action in target cells, i.e., insulin resistance, are both observed. However, it is not yet clear which of these changes has primary importance.

Preventing complications by administering an excellent hypoglycemic agent at an early stage of onset is known as the most effective method of treating diabetes. Oral hypoglycemic agents for diabetes treatment include sulfonylureas, biguanides, and acarbose. Among these agents, in the case of sulfonylurea preparations, second-generation products with a short action time and strong blood glucose lowering effect are mainly used. For non-obese adult type diabetes patients, biguanide drugs are used. Acarbose is used in diabetic patients who are overweight and obese. One of the biggest differences between biguanide and acarbose from sulfonylurea preparations is that the frequency of incidence of hypoglycemia is low.

An ideal hypoglycemic agent is a safe oral drug that may correct metabolic abnormalities specific to diabetes, and which takes effect quickly to prevent postprandial blood glucose increase and also loses its efficacy within a short period of time so as to prevent unnecessary hypoglycemia. Recently, the development of such a hypoglycemic agent is in high demand, and a method of treating diabetes using traditional medicines or natural materials is in the spotlight.

Currently, in the case of therapeutic agents for non-insulin-dependent diabetes, efforts are being made to develop a therapeutic agent which has reduced side effects while being able to treat insulin-dependent and non-insulin-dependent diabetes. Traditionally, herbal medicines used in oriental medicine are known to have low toxicity and various therapeutic effects. Until now, single or combined prescriptions using dozens of herbal medicines have been reported as having therapeutic effects on diabetes and as being able to treat insulin-dependent and non-insulin-dependent diabetes at the same time.

Ginger (Zingiber officinale) is a rhizome of a perennial herbaceous plant of the ginger family, cultivated in tropical and subtropical regions such as Egypt and Iraq since prehistoric times. Ginger is a spice that is widely used around the world because of its unique aroma and taste. Ginger began to be cultivated in Korea in the 1930s. Ginger was mainly cultivated in South Jeolla Province in the early days, but has been gradually cultivated nationwide due to improved cultivation conditions and climate change. Currently, the ginger production in North Gyeongsang and Chungcheong Province accounts for more than 85% of Korea's ginger production. In oriental medicine and folk medicine, dried ginger has mainly been used. Ginger stimulates metabolism and relieves sweat and phlegm, as well as regulate blood circulation and body temperature when eaten. Therefore, ginger has been used as it is known to be effective in fever, cold, and flu. According to the Dongui Bogam, ginger is ‘warm in nature, tasty, and non-toxic’. It is known that ginger enters the zang-fu organs, relieves phlegm, lowers energy, and stops vomiting. In addition, ginger is known to treat chills and dampness, hiccups, shortness of breath and coughing, as well as boost energy. In addition, according to Shennong Bencaojing, when one consumes ginger continually, they will gain divine power. Ginger has also been used in oriental medicine as an aromatic, a stomach medicine, a flavoring agent, and a treatment for nausea. Ginger also has a diuretic effect, and thus, ginger has been known to be effective in reducing swelling by effectively inducing sweating and urinating. According to Jeong Yak-yong's Dasanbang, ginger juice was used for stroke. According to the herbalist Shi-Jin Lee's Compendium of Materia Medica, hot ginger juice or ginger soup is effective for neuralgia, arthritis, frostbite, or the like, and ginger was used to enhance the drug delivery effect by adding ginger to various herbal prescriptions due to having properties that facilitate the absorption of drugs.

Gingerol and shogaol components, which give ginger's unique pungent taste, have various medicinal effects, and the structure of these components may be represented by Formula 1 and Formula 2, respectively. When n=4, 6, or 8, Formula 1 may be 4-gingerol, 6-gingerol, or 8-gingerol, respectively. When n=4, 6, or 8, Formula 2 may be 4-shogaol, 6-shogaol, or 8-shogaol, respectively:

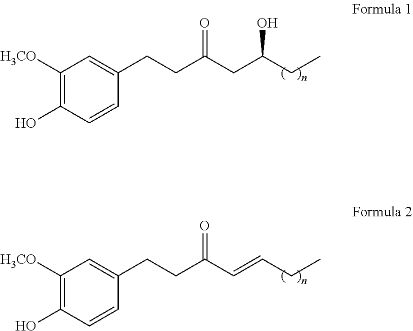

Gingerol has a vanillyl ketone structure that is important in exhibiting anticancer effects. As the shogaol content is very small ginger, research on shogaol has been relatively neglected as compared with gingerol. However, it is known that the anticancer activity of shogaol through induction of cell defense mechanisms is more than 10 times higher than that of [6]-gingerol, which has the highest content. [6]-shogaol is effective in cardiovascular disease, gastrointestinal system, and vomiting, in addition to its anticancer effects. [6]-shogaol is also known to have anti-inflammatory effects, antioxidant effects, antithrombotic effects, neuroprotective effects, anti-obesity effects, and learning disability and memory enhancement effects.

The present inventors developed a method of microwave-processing under pressure for a simple ginger extract, to increase [6]-shogaol, [8]-shogaol, and [10]-shogaol by dehydration of [6]-gingerol, [8]-gingerol, and [10]-gingerol contained in ginger (KR 10-1514793). Further, the present invention was completed by discovering that ginger extract and shogaols processed by microwaves under pressure have the effect of stimulating glucose-dependent insulin secretion.

### SUMMARY

One or more embodiments include an antidiabetic composition including a ginger extract, as an active ingredient, obtained by microwave irradiation, under pressure, of ginger, an extract thereof, or a combination thereof, in which a content of [6]-shogaol, [8]-shogaol, and [10]-shogaol may be 20 wt % or more greater than a content of [6]-gingerol, [8]-gingerol, and [10]-gingerol, wherein the microwave irradiation may be performed under a pressure in a range of about 2 atm to about 100 atm.

One or more embodiments include a method of preventing or treating diabetes in a subject, the method including administering the antidiabetic pharmaceutical composition to a subject.

Additional aspects will be set forth in part in the description which follows and, in part, will be apparent from the description, or may be learned by practice of the presented embodiments of the disclosure.

One or more embodiments include an antidiabetic composition including a ginger extract, as an active ingredient, obtained by microwave irradiation, under pressure, of ginger, an extract thereof, or a combination thereof, in which a content of [6]-shogaol, [8]-shogaol, and [10]-shogaol may be 20 wt % or more greater than a content of [6]-gingerol, [8]-gingerol, and [10]-gingerol, wherein the microwave irradiation may be performed under a pressure in a range of about 2 atm to about 100 atm.

For the antidiabetic composition, the microwave irradiation may be performed according to the method described in KR 10-1514793. The extract used in the microwave irradiation may be extracted by using a solvent after the microwave irradiation.

For the antidiabetic composition, the microwave irradiation may be performed at a temperature of about 120° C. to about 200° C. for about 30 minutes to about 90 minutes. The microwave irradiation may be performed in the presence of an acidic substance. The acidic substance may be acetic acid, lysine, leucine, or a combination thereof.

For the antidiabetic composition, the extract used in the microwave irradiation may be a crude extract ginger from water, C₁-C₄ alcohol, or a mixture thereof; a solvent fraction of n-hexane, methylene chloride, ethyl acetate, butanol, methanol, acetone, or a mixture thereof, of the crude extract; or a purified product of the solvent fraction.

In the antidiabetic composition, in the ginger extract, a content of [6]-shogaol, [8]-shogaol, and [10]-shogaol may be 80 wt % or greater of a total content of [6]-shogaol, [8]-shogaol, [10]-shogaol, [6]-gingerol, [8]-gingerol, and [10]-gingerol. In the ginger extract, a content of [6]-shogaol may be 80 wt % or greater of a total content of [6]-shogaol, [8]-shogaol, [10]-shogaol, [6]-gingerol, [8]-gingerol, and [10]-gingerol. In the ginger extract, a content of [6]-shogaol, [8]-shogaol, and [10]-shogaol may be 80 wt % or greater of a total content of [6]-shogaol, [8]-shogaol, [10]-shogaol, [6]-gingerol, [8]-gingerol, and [10]-gingerol.

In the antidiabetic composition, in the ginger extract, a content of [6]-shogaol, [6]-gingerol, and [8]-gingerol may be in a range of about 7.5 wt % to about 12.5 wt %; about 2.5 wt % to about 7.5 wt %; or about 80 wt % to about 90 wt %, based on a total content of [6]-shogaol, [8]-shogaol, [10]-shogaol, [6]-gingerol, [8]-gingerol, and [10]-gingerol.

In the antidiabetic composition, the ginger extract may be a fraction obtained by fractionating the crude extract with ethanol. In the fraction of the ginger extract, a content of [6]-shogaol, [6]-gingerol, and [8]-gingerol may be in a range of about 7.5 wt % to about 12.5 wt %; about 2.5 wt % to about 7.5 wt %; or about 80 wt % to about 90 wt %, based on a total content of [6]-shogaol, [8]-shogaol, [10]-shogaol, [6]-gingerol, [8]-gingerol, and [10]-gingerol. In addition, in the fraction of the ginger extract, a content of [6]-shogaol, [8]-shogaol, and [10]-shogaol may be 80 wt % or greater of a total content of [6]-shogaol, [8]-shogaol, [10]-shogaol, [6]-gingerol, [8]-gingerol, and [10]-gingerol. In the fraction of the ginger extract, a content of [6]-shogaol may be 80 wt % or greater of a total content of [6]-shogaol, [8]-shogaol, [10]-shogaol, [6]-gingerol, [8]-gingerol, and [10]-gingerol. In the fraction of the ginger extract, a content of [6]-shogaol, [8]-shogaol, and [10]-shogaol may be 80 wt % or greater of a total content of [6]-shogaol, [8]-shogaol, [10]-shogaol, [6]-gingerol, [8]-gingerol, and [10]-gingerol. The ethanol may be an about 70% to about 90% aqueous solution.

After the microwave irradiation, a processed ginger product with an increased shogaol rate may be obtained by removing the solvent by distillation under reduced pressure or freeze-drying. The distillation under reduced pressure may be performed, for example, at a temperature in a range of about 10° C. to about 40° C., or about 20° C. to about 30° C. The freeze-drying may be performed according to a general drying method.

In addition, a raw ginger extract and a microwave-processed ginger product may each be a fraction separated by chromatography or a purified single component, depending on the ingredients contained.

The fraction of the raw ginger extract and the microwave-processed ginger product may be obtained by a reversed-phase silica gel flash chromatography method. In some embodiments, Octadecyl-silica (C18 ODS) may be filled into an open column, and 20%, 40%, 60%, 80% ethanol aqueous solution, 100% ethanol, and 100% acetone may each be sequentially flowed for the raw ginger extract or the microwave-processed ginger product. Then, an eluent is received and the fraction may be obtained by distillation under reduced pressure.

Chromatography used in obtaining a single component by separating and purifying the raw ginger extract and the microwave-processed ginger product by chromatography may be, for example, reverse-phase preparative high-performance liquid chromatography (Prep-HPLC).

Separation conditions by Prep-HPLC may vary depending on an amount of sample and a size of a tube used. In general, a reversed-phase preparative column (C18(2) column available from Phenomenex Luna, particle size of 10 μm, column size of 250×21.20 mm) may be prepared in a liquid chromatography apparatus (available from Gilson Companion), and the raw ginger extract or the microwave-processed ginger product may be dissolved in an initial eluent to inject the solution thereto. Then, the eluent may be developed by using a solution of methanol:water 50:50 to 100:0 for 60 minutes to 90 minutes for separation.

In the antidiabetic composition, the antidiabetic composition may promote insulin secretion ability of cells.

The ginger extract may be a fraction obtained by fractionating a crude ginger extract with C₁-C₆ alcohols such as methanol and ethanol. The methanol or ethanol may be an about 70% to about 90% aqueous solution.

The antidiabetic composition may be a pharmaceutical composition or a food composition. The pharmaceutical composition may be for preventing or treating diabetes. The food composition may be for preventing or ameliorating diabetes.

The composition may include a pharmaceutically or food-acceptable carrier or diluent. The carrier may be an excipient, preservative, stabilizer, wetting agent or emulsifying agent, salt for regulating osmotic pressure, buffer, or other material useful for preventing, treating or ameliorating diabetes.

The composition may be an orally administered agent or a parenteral dosage form. The orally administered agent may be, for example, tablets, pills, hard and soft capsules, solutions, suspensions, emulsions, syrups, powders, fine granules, granules, or pellets. These formulations may contain, in addition to an active ingredient, surfactants, diluents (e.g., lactose, dextrose, sucrose, mannitol, sorbitol, cellulose, and glycine), lubricants (e.g., silica, talc, stearic acid and magnesium or calcium salts thereof, and polyethylene glycol). Tablets may also contain binders such as magnesium aluminum silicate, starch paste, gelatin, tragacanth, methylcellulose, sodium carboxymethylcellulose, and polyvinyl pyrrolidine. In addition, in some cases, the tablets may contain pharmaceutical or food additives, such as starch, agar, a disintegrant such as alginic acid or a sodium salt thereof, absorbents, colorants, flavoring agents, and sweeteners. The tablets may be prepared by general mixing, granulation, or coating methods. In addition, the parenteral dosage form may be a transdermal dosage form, for example, injections, drops, ointments, lotions, gels, creams, sprays, suspensions, emulsions, suppositories, patches, or the like.

The pharmaceutical composition may be administered, for example, parenterally, rectally, topically, transdermally, or subcutaneously. In addition, the pharmaceutical composition may be, for example, administered topically to the scalp.

The dosage of the active ingredient may be determined within a level by one of ordinary skill in the art, and a dosage of drug per day may vary depending on various factors such as a degree of diabetes progression, onset time, age, health status, complications, or the like of the subject to which the pharmaceutical composition is to be administered. The dosage to be administered for adults may be, for example, the composition in a range of about 1 μg/kg to about 200 mg/kg or about 50 μg/kg to about 50 mg/kg in divided doses 1 to 3 times a day.

The food composition may be any food including a ginger extract, as an active ingredient, obtained by microwave irradiation, under pressure, of ginger, an extract thereof, or a combination thereof, in which a content of [6]-shogaol, [8]-shogaol, and [10]-shogaol may be 20 wt % or more greater than a content of [6]-gingerol, [8]-gingerol, and [10]-gingerol, wherein the microwave irradiation may be performed under a pressure in a range of about 2 atm to about 100 atm. The food may be in a form of dairy products, gum, confectionery, or drinking liquid.

One or more embodiments include a method of preventing or treating diabetes in a subject, the method including administering the pharmaceutical composition to a subject. The subject may be a subject suffering from diabetes or with a high possibility of having diabetes. The subject may be a mammal. The subject may be a human, a pig, a cat, a dog, or a cow. The administration may be performed to an effective amount to prevent or treat diabetes. The administration may be oral or parental administration. The parenteral administration may be performed by injection through the skin. The dosage may vary depending on various factors such as a degree of diabetes progression, onset time, age, health status, complications, or the like of the subject to which the pharmaceutical composition is to be administered. For example, the dosage to be administered for adults may be, for example, the composition in a range of about 1 μg/kg to about 200 mg/kg or about 50 μg/kg to about 50 mg/kg in divided doses 1 to 3 times a day.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A to 1G show reverse-phase chromatography results of a ginger ethanol extract in 20% ethanol, 40% ethanol, 60% ethanol, 80% ethanol, 100% ethanol, and 100% acetone fractions;

FIGS. 2A to 2G show results of reverse-phase chromatography of a ginger extract obtained by microwave irradiation under pressure of a ginger ethanol extract in 20% ethanol, 40% ethanol, 60% ethanol, 80% ethanol, 100% ethanol, and 100% acetone fractions;

FIG. 3 is a graph of results of an oral glucose tolerance test of a ginger extract and a microwave-processed ginger product; and

FIG. 4 is graph of results measured by oral glucose tolerance test by collecting blood from a caudal vein 15, 30, 60, and 120 minutes after oral administration of glucose to investigate the effect of administration of a single ingredient contained in ginger on oral glucose tolerance.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

The present disclosure will be described in more detail through the following Examples. However, these Examples are for illustrative purposes only, and the present disclosure is not intended to be limited by these Examples.

### Example 1: Preparation of Processed Ginger Product by Simple Heat-Drying

A processed ginger product was prepared from ginger through simple processing as follows. Specifically, 100 g of undried ginger was cut and steamed at a temperature of 100° C. for 6 hours and 12 hours, and then dried to obtain steamed ginger. 800 mL of 80% by volume ethanol aqueous solution was added to the simple-processed ginger as described above, and the mixture was refluxed and extracted at a temperature of 70° C. for 3 hours. Then, the solvent was evaporated under reduced pressure, followed by drying of the residue to thereby obtain a simple-processed ginger extract in powder form.

### Example 2: Preparation of Microwave-Processed Ginger Product Under Reduced Pressure

The processing in Example 2 was performed in the same manner as the ginger processing method for increasing a shogaol content by microwave irradiation under pressure of a simple ginger extract according to KR 10-1514793.

Specifically, 10 g of raw ginger dry extract was added to 50 mL of a 50 vol % aqueous solution of ethanol in an 80 mL container of a pressurized microwave irradiator (CEM, Model No. 909150, USA). The container was sealed, and the processing temperature was set to a temperature of 140° C. Microwaves were irradiated for 30 minutes at 100 W (frequency of 2,455 MHz) in a fixed state. After completion of processing, lyophilization was performed to obtain microwave-processed ginger product in powder form. The pressure during microwave irradiation was 6 bar.

### Example 3: Preparation of Fraction of Ginger Extract

The raw ginger fraction used in Example 2 was obtained by a reversed-phase silica gel flash chromatography method.

Specifically, octadecyl-silica (C18 ODS) was filled into a glass column having a diameter of 3 cm, to a height of 20 cm, and 2 g of the raw ginger extract, i.e., the ginger extract obtained in Example 1, was added to a specific thickness. After sequentially flowing 200 mL of each of 20%, 40%, 60%, and 80% ethanol aqueous solutions, 100% ethanol, and 100% acetone thereto, the eluent was distilled under reduced pressure to obtain fractions of 840 mg, 233 mg, 225 mg, 115 mg, 131 mg, and 49 mg, respectively.

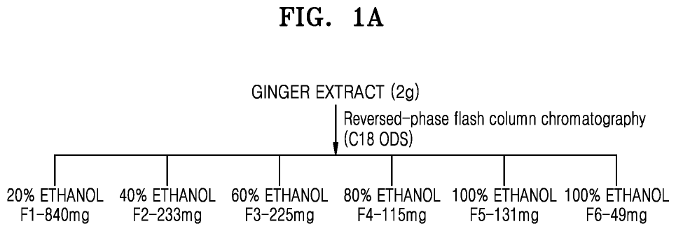

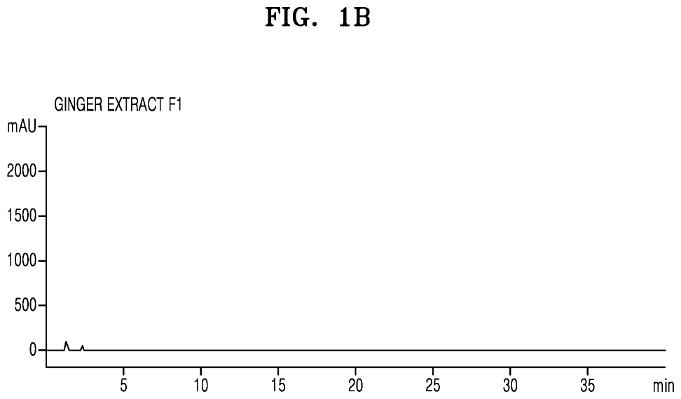

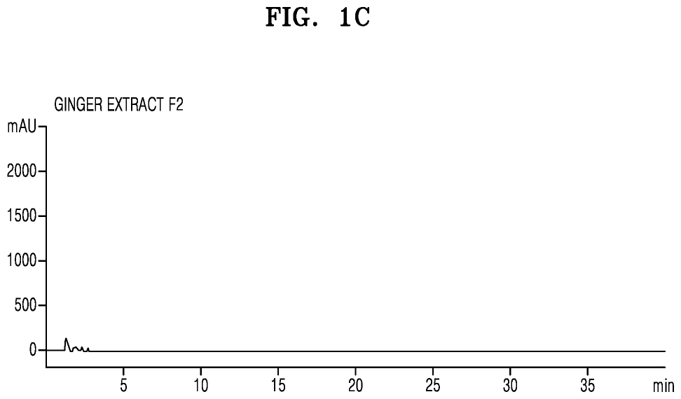

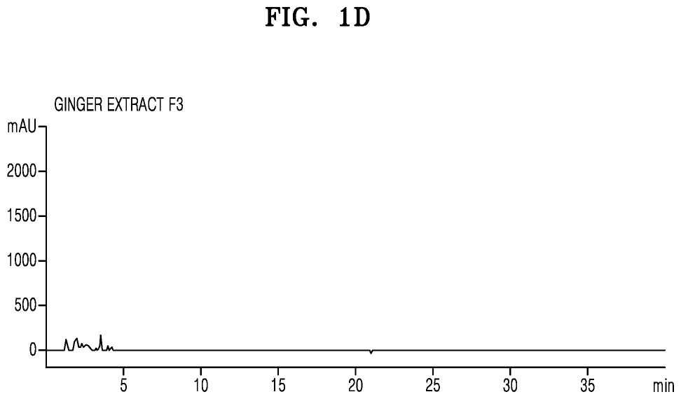

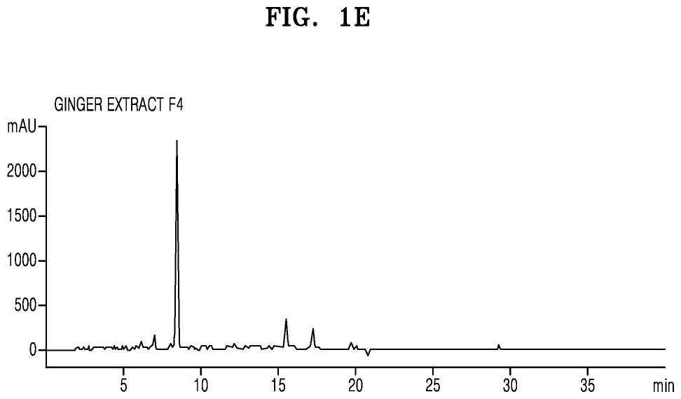

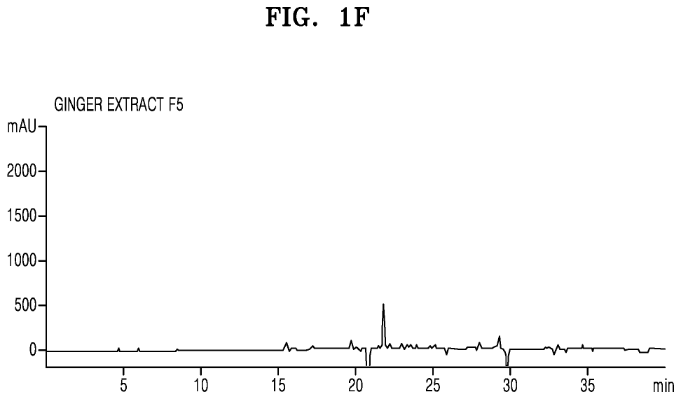

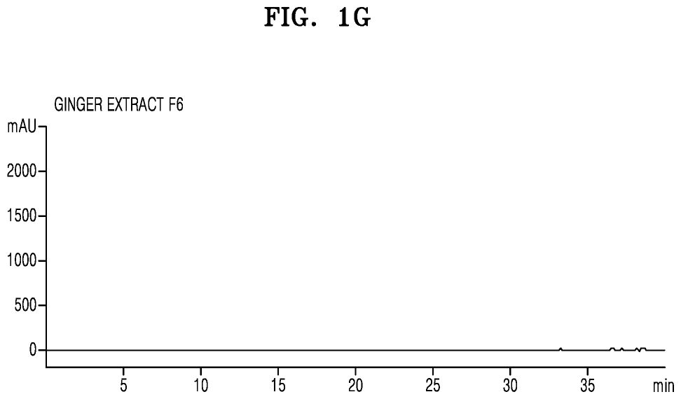

FIGS. 1A to 1G show reverse-phase chromatography results of the ginger ethanol extract in 20% ethanol, 40% ethanol, 60% ethanol, 80% ethanol, 100% ethanol, and 100% acetone fractions.

### Example 4: Preparation of Fraction of Processed Ginger Product

The microwave-processed ginger fraction used in Example 4 was obtained by using the processed ginger product prepared in Example 2 through a reversed-phase silica gel flash chromatography method.

Specifically, octadecyl-silica (C18 ODS) was filled into a glass column having a diameter of 3 cm, to a height of 20 cm, and 2 g of the microwave-processed ginger product was added to a specific thickness. After sequentially flowing 200 mL of each of 20%, 40%, 60%, and 80% ethanol aqueous solutions, 100% ethanol, and 100% acetone thereto, the eluent was distilled under reduced pressure to obtain fractions of 428 mg, 323 mg, 369 mg, 145 mg, 167 mg, and 67 mg, respectively.

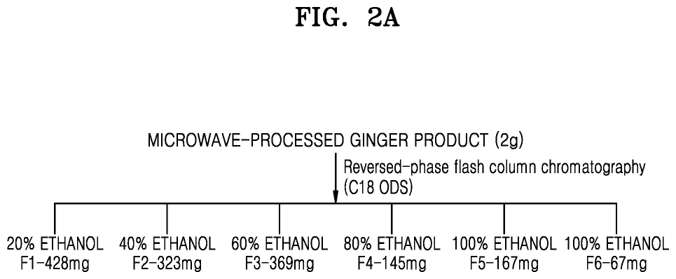

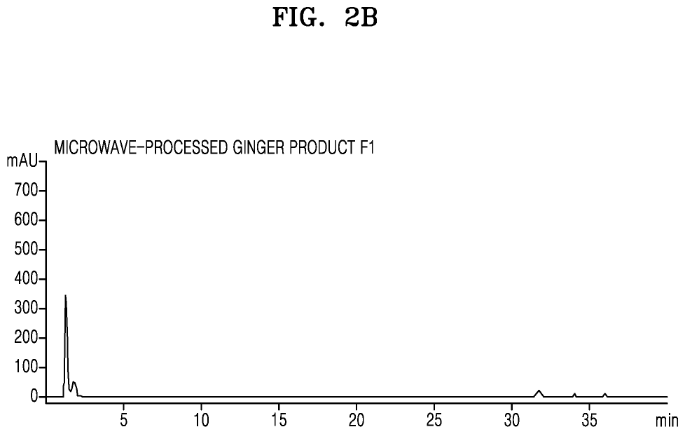

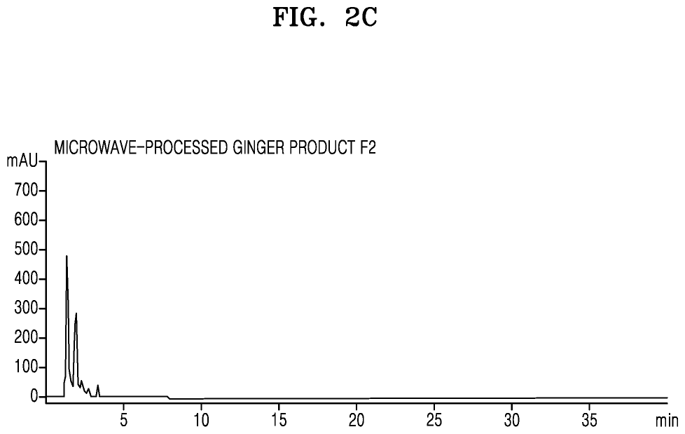

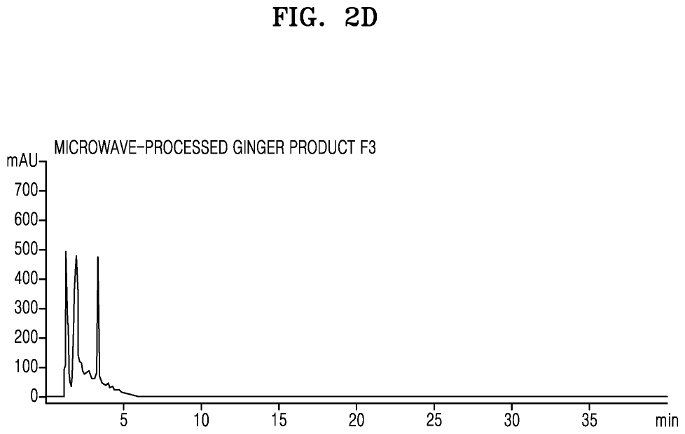

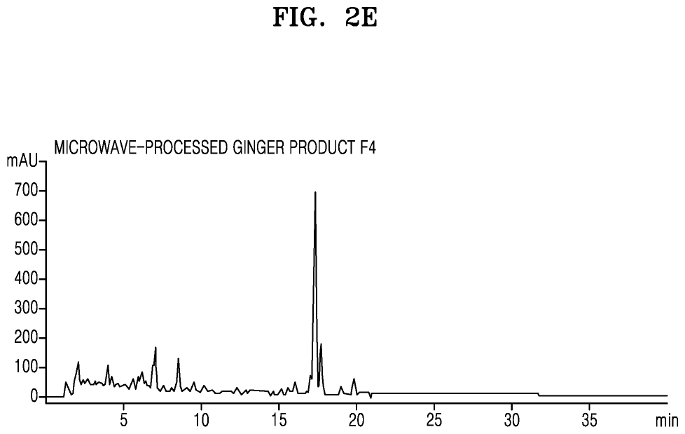

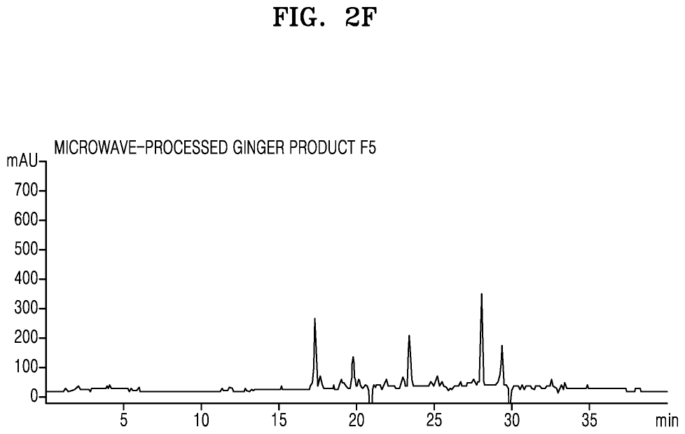

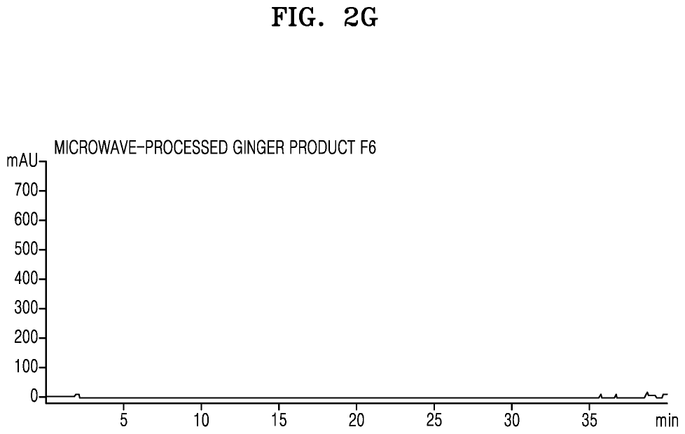

FIGS. 2A to 2G show reverse-phase chromatography results of a ginger extract obtained by microwave irradiation under pressure of a ginger ethanol extract in 20% ethanol, 40% ethanol, 60% ethanol, 80% ethanol, 100% ethanol, and 100% acetone fractions.

### Example 5: Preparation of Single Components of [6]-Gingerol, [8]-Gingerol, [10]-Gingerol, [6]-Shogaol, [8]-Shogaol, and [10]-Shogaol

From the ginger extracts and the microwave-processed ginger products of Examples 1 and 2, [6]-gingerol, [8]-gingerol, [10]-gingerol, [6]-shogaol, [8]-shogaol, and [10]-shogaol compounds were separated.

Through Prep-HPLC (stationary phase: Phenomenex Luna C18(2) column, particle size of 10 μm, a column size of 250×21.20 mm), the eluent was developed from 30% to 100% of initial acetonitrile for 60 to 90 minutes at a flow rate of 8 mL/min to thereby obtain 6 peaks appearing at UV of 282 nm. For Peaks 1 to 6, through reversed-phase semi-preparative high-performance liquid chromatography (stationary phase: Phenomenex Gemini 6 Phenyl column, particle size of 5 μm, column size of 250×10 mm), the eluent was developed from 50% to 100% of initial methanol for 60 to 90 minutes at a flow rate of 4 mL/min to thereby obtain [6]-gingerol, [8]-gingerol, [10]-gingerol, [6]-shogaol, [8]-shogaol, and [10]-shogaol compounds.

The compounds were identified through nuclear magnetic resonance spectrometer (NMR), mass spectroscopy, or the like, and [6]-gingerol, [8]-gingerol, [10]-gingerol, [6]-shogaol, [8]-shogaol, and [10]-shogaol were confirmed by referring to existing literature information. Chemical formulae of the separated compounds are the same as Formula 1 and Formula 2 above.

### Experimental Example 1: In Vitro Insulin Secretion Evaluation of Ginger Extract and Fraction Thereof, Steamed Ginger Extract, Microwave-Processed Ginger Product and Fraction Thereof, and Single Ingredients

1. Experiment Method

Insulin secretion ability was evaluated using a mouse insulinoma cell line INS-1 (Biohermes, Shanghai, China) as follows.

(1) INS-1 Cell Culture

Cell line INS-1 (Biohermes, Shanghai, China) is a mouse insulinoma species and has insulin secretion ability. INS-1 cells were cultured in RPMI1640 (Cellgro, Manassas, Va., USA) medium supplemented with 10% fetal bovine serum (FBS), 100 units/mL penicillin G, 100 μg/mL streptomycin, 10 mM HEPES, 2 mM L-glutamine, 1 mM sodium pyruvate and 0.05 mM 2-mercapto ethanol under a condition of a temperature of 37° C., 90% air, and 10% CO₂. Among the above-mentioned components, reagents not mentioned by the manufacturer were purchased from Gibco BRL, Life Technologies.

(2) Measurement of INS-1 Cell Toxicity

The cultured INS-1 cells were inoculated in each well of a 96-well plate in the number of 1×10⁴ in a medium of 100 μL and cultured for 24 hours to stabilize the cells. Next, the ginger extract, fraction thereof, and single compounds were added to a specific concentration, followed by culturing for 24 hours. Next, 10 μL of a CCK-8 reagent (Dojindo Laboratories, Japan) was added to each well, followed by culturing at a temperature of 37° C. After 1 hour, cell viability was measured by measuring absorbance at a wavelength of 450 nm on a BIO-TEK (Winooski, Vt., USA) microplate reader. The specific concentrations of the ginger extract and a fraction thereof were set to 1 μg/m L, 2.5 μg/mL, and 5 μg/mL, and the specific concentrations of the single compound were set to 1 μM, 2.5 μM, and 5 μM.

Table 1 shows toxicity test results of the ginger extract, an extract of ginger steamed for 6 hours, and an extract of ginger steamed for 12 hours, the fraction of the ginger extract, the microwave-processed ginger product, and the fraction thereof for INS-1 cells, which are mouse insulinoma cells.

TABLE 1

| Classification | Concentration (μg/mL) | Cell viability (%) |
|---|---|---|
| Ginger extract | 1 | 99.5 ± 2.0 |
| | 2.5 | 97.8 ± 0.2 |
| | 5 | 98.4 ± 3.7 |
| 6 hours-steamed ginger | 1 | 101.5 ± 1.3 |
| | 2.5 | 98.8 ± 1.7 |
| | 5 | 95.3 ± 1.2 |
| 12 hours-steamed ginger | 1 | 97.4 ± 2.7 |
| | 2.5 | 98.4 ± 3.3 |
| | 5 | 98.6 ± 3.2 |
| Microwave-processed | 1 | 97.0 ± 4.8 |
| ginger product | 2.5 | 101.4 ± 3.5 |
| | 5 | 102.8 ± 3.3 |
| Ginger extract F1 | 1 | 104.3 ± 4.8 |
| | 2.5 | 101.0 ± 3.3 |
| | 5 | 101.7 ± 2.9 |
| Ginger extract F2 | 1 | 102.7 ± 2.0 |
| | 2.5 | 102.7 ± 4.8 |
| | 5 | 101.9 ± 3.3 |
| Ginger extract F3 | 1 | 91.6 ± 1.8 |
| | 2.5 | 93.8 ± 3.1 |
| | 5 | 102.5 ± 0.3 |
| Ginger extract F4 | 1 | 105.4 ± 2.7 |
| | 2.5 | 100.3 ± 2.6 |
| | 5 | 103.5 ± 1.0 |
| Ginger extract F5 | 1 | 99.9 ± 2.7 |
| | 2.5 | 100.3 ± 3.0 |
| | 5 | 101.3 ± 2.0 |
| Ginger extract F6 | 1 | 99.4 ± 2.1 |
| | 2.5 | 100.0 ± 2.5 |
| | 5 | 100.0 ± 2.1 |
| Microwaved-processed | 1 | 102.3 ± 1.0 |
| ginger product F1 | 2.5 | 103.5 ± 3.5 |
| | 5 | 105.6 ± 3.6 |
| Microwaved-processed | 1 | 98.6 ± 3.6 |
| ginger product F2 | 2.5 | 97.0 ± 0.4 |
| | 5 | 99.9 ± 3.3 |
| Microwaved-processed | 1 | 101.3 ± 4.1 |
| ginger product F3 | 2.5 | 99.1 ± 3.2 |
| | 5 | 98.5 ± 4.3 |
| Microwaved-processed | 1 | 107.3 ± 4.5 |
| ginger product F4 | 2.5 | 100.2 ± 3.8 |
| | 5 | 102.4 ± 4.5 |
| Microwaved-processed | 1 | 105.6 ± 3.8 |
| ginger product F5 | 2.5 | 100.5 ± 4.2 |
| | 5 | 98.8 ± 2.7 |
| Microwaved-processed | 1 | 96.5 ± 3.7 |
| ginger product F6 | 2.5 | 97.3 ± 3.2 |
| | 5 | 102.3 ± 3.8 |

Table 2 shows toxicity test results of [6]-gingerol, [8]-gingerol, [10]-gingerol, [6]-shogaol, [8]-shogaol, and [10]-shogaol, which are single compounds contained in ginger, and gliclazide, for an INS-1 cell, which is a mouse insulinoma cell.

TABLE 2

| Classification | Concentration (μM) | Cell viability (%) |
|---|---|---|
| [6]-gingerol | 1 | 98.1 ± 2.7 |
| | 2.5 | 98.7 ± 2.3 |
| | 5 | 98.2 ± 3.5 |
| [8]-gingerol | 1 | 97.4 ± 1.0 |
| | 2.5 | 96.1 ± 2.2 |
| | 5 | 96.4 ± 1.4 |
| [10]-gingerol | 1 | 99.3 ± 1.6 |
| | 2.5 | 100.6 ± 3.3 |
| | 5 | 100.7 ± 3.5 |
| [6]-shogaol | 1 | 101.1 ± 0.4 |
| | 2.5 | 101.8 ± 0.7 |
| | 5 | 101.2 ± 0.2 |
| [8]-shogaol | 1 | 94.0 ± 2.6 |
| | 2.5 | 96.3 ± 1.9 |
| | 5 | 92.4 ± 1.7 |
| [10]-shogaol | 1 | 101.9 ± 2.2 |
| | 2.5 | 99.8 ± 3.9 |
| | 5 | 99.6 ± 3.3 |
| Glyclazide | 1 | 99.7 ± 0.2 |
| | 2.5 | 96.3 ± 0.9 |
| | 5 | 91.1 ± 1.4 |

Cell viability in Tables 1 and 2 was calculated as follows.

Cell viability (%)=A/B×100

A: absorbance of sample to which a reagent is not added

B: absorbance of sample to which a reagent is added

As shown in Tables 1 and 2, the ginger extract, the extract ginger steamed for 6 hours, the extract ginger steamed for 12 hours, the fraction of the ginger extract, the microwave-processed ginger product and a fraction thereof, single compounds contained in ginger, such as [6]-gingerol, [8]-gingerol, [10]-gingerol, [6]-shogaol, [8]-shogaol, and [10]-shogaol, were found not to exhibit toxicity against INS-1 cells.

(3) Measurement of Ability of Glucose Stimulated Insulin Secretion (GSIS) of INS-1 Cell

5×10⁵ cultured INS-1 cells were inoculated in each well of the 12-well plate in the same medium of 2 mL and cultured for 24 hours under the same condition as described in Section (1) INS-1 cell culture to stabilize the cells. Then, 114 mM sodium chloride (NaCl), 4.4 mM potassium chloride (KCl), 1 mM magnesium sulfate (MgSO₄), 1.28 mM calcium chloride (CaCl₂)), 29.5 mM sodium hydrogen carbonate (NaHCO₃), 10 mM HEPES; Gibco BRL Life Technologies), and 0.1% bovine serum albumin (BSA) were mixed, and the cells were washed twice with 2 mL of Krebs-Ringer buffer with a hydrogen ion concentration of pH 7.4. Thereafter, 2 mL of Krebs-Ringer buffer was added to the washed cells and incubated for 1 hour. Then, the ginger extract, each fraction, and each compound were incubated for 30 minutes in 1.8 mL of Krebs-Ringer buffer at a specific concentration. The specific concentrations of the ginger extract and a fraction thereof were set to 1 μg/m L, 2.5 μg/mL, and 5 μg/mL, and the specific concentrations of the single compound were set to 1 μM, 2.5 μM, and 5 μM.

0.2 mL of Krebs-Ringer buffers containing 33 mM and 167 mM glucose were each added to the wells and incubated for 1 hour. Thereafter, after centrifugation at 12,000 revolutions per minute (rpm) for 10 minutes at a temperature of 4° C., the supernatant was taken, and the amount of insulin was measured with a Rat insulin RIA kit (Gentaur Molecular Products, Belgium). The stimulation index (SI) is a value obtained by dividing a value measured at a high glucose concentration, that is, the amount of insulin secreted by stimulation of a 16.7 mM glucose concentration by a value measured at a low glucose concentration, that is, the amount of insulin secreted by stimulation of a 3.3 mM glucose concentration. The measured SIs are shown in Tables 3 and 4. As a positive control, gliclazide was used at concentrations of 2.5 μM, 5.0 μM, 10 μM, and 20 μM.

Table 3 shows insulin secretion ability test results of the ginger extract, the extract of ginger steamed for 6 hours, and the extract of ginger steamed for 12 hours, the fraction of the ginger extract, the microwave-processed ginger product, and the fraction thereof for INS-1 cells, which are mouse insulinoma cells.

TABLE 3

| Classification | Concentration (μg/mL) | Stimulation index (SI) |
|---|---|---|
| Ginger extract | 1 | 5.1 ± 0.1 |
| | 2.5 | 7.7 ± 0.2 |
| | 5 | 6.4 ± 0.4 |
| 6 hours-steamed ginger | 1 | 6.1 ± .02 |
| | 2.5 | 3.7 ± 0.0 |
| | 5 | 2.5 ± 0.1 |
| 12 hours-steamed ginger | 1 | 2.2 ± 0.5 |
| | 2.5 | 2.7 ± 0.0 |
| | 5 | 4.2 ± 0.0 |
| Microwave-processed | 1 | 6.5 ± 0.6 |
| ginger product | 2.5 | 6.0 ± 0.2 |
| | 5 | 12.4 ± 0.4 |
| Ginger extract F1 | 1 | 1.2 ± 0.0 |
| | 2.5 | 1.4 ± 0.1 |
| | 5 | 4.3 ± 0.3 |
| Ginger extract F2 | 1 | 2.3 ± 0.0 |
| | 2.5 | 2.9 ± 0.1 |
| | 5 | 6.2 ± 0.1 |
| Ginger extract F3 | 1 | 3.2 ± 0.3 |
| | 2.5 | 2.3 ± 0.0 |
| | 5 | 2.7 ± 0.1 |
| Ginger extract F4 | 1 | 1.3 ± 0.0 |
| | 2.5 | 2.8 ± 0.1 |
| | 5 | 4.1 ± 0.0 |
| Ginger extract F5 | 1 | 5.7 ± 0.1 |
| | 2.5 | 6.7 ± 0.3 |
| | 5 | 10.8 ± 0.6 |
| Ginger extract F6 | 1 | 2.4 ± 0.0 |
| | 2.5 | 3.1 ± 0.0 |
| | 5 | 3.9 ± 0.2 |
| Microwaved-processed | 1 | 3.1 ± 0.1 |
| ginger product F1 | 2.5 | 4.5 ± 0.0 |
| | 5 | 5.4 ± 0.0 |
| Microwaved-processed | 1 | 4.9 ± 0.0 |
| ginger product F2 | 2.5 | 6.2 ± 0.2 |
| | 5 | 7.4 ± 0.0 |
| Microwaved-processed | 1 | 1.2 ± 0.0 |
| ginger product F3 | 2.5 | 1.5 ± 0.0 |
| | 5 | 6.4 ± 0.1 |
| Microwaved-processed | 1 | 2.1 ± 0.0 |
| ginger product F4 | 2.5 | 8.5 ± 1.1 |
| | 5 | 18.1 ± 0.5 |
| Microwaved-processed | 1 | 2.3 ± 0.0 |
| ginger product F5 | 2.5 | 3.4 ± 0.2 |
| | 5 | 3.0 ± 0.0 |
| Microwaved-processed | 1 | 1.0 ± 0.0 |
| ginger product F6 | 2.5 | 5.0 ± 0.0 |
| | 5 | 5.3 ± 0.1 |

Table 4 shows insulin secretion ability test results of [6]-gingerol, [8]-gingerol, [10]-gingerol, [6]-shogaol, [8]-shogaol, and [10]-shogaol, which are single compounds contained in ginger, and gliclazide for INS-1 cells, which are mouse insulinoma cells.

TABLE 4

| Classification | Concentration (μM) | Stimulation index (SI) |
|---|---|---|
| [6]-gingerol | 1 | 2.4 ± 0.0 |
| | 2.5 | 2.2 ± 0.1 |
| | 5 | 2.3 ± 0.1 |
| [8]-gingerol | 1 | 1.1 ± 0.0 |
| | 2.5 | 1.5 ± 0.0 |
| | 5 | 2.1 ± 0.0 |
| [10]-gingerol | 1 | 1.7 ± 0.2 |
| | 2.5 | 1.7 ± 0.1 |
| | 5 | 1.5 ± 0.1 |
| [6]-shogaol | 1 | 3.0 ± .0 |
| | 2.5 | 3.5 ± 0.1 |
| | 5 | 4.8 ± 0.0 |
| [8]-shogaol | 1 | 1.3 ± 0.1 |
| | 2.5 | 1.5 ± 0.1 |
| | 5 | 1.8 ± 0.1 |
| [10]-shogaol | 1 | 1.9 ± 0.0 |
| | 2.5 | 1.4 ± 0.0 |
| | 5 | 1.4 ± 0.1 |
| Glyclazide | 1 | 1.3 ± 0.0 |
| | 2.5 | 1.9 ± 0.1 |
| | 5 | 3.0 ± 0.1 |

Table 5 shows results of measuring the content of a single component using ultra performance liquid chromatography (UPLC) for processed ginger products F1 to F6. Each single component was prepared at 10 ppm, 25 ppm, 50 ppm, 100 ppm, and 250 ppm, and a verification line was prepared after UPLC analysis. After that, by analyzing the fraction of the processed ginger product according to the same method, the content of each of [6]-gingerol, [8]-gingerol, [10]-gingerol, [6]-shogaol, [8]-shogaol, and [10]-shogaol was analyzed The results thereof are shown in Table 5 as mg per 1 g of the fraction.

TABLE 5

| | [6]- | [8]- | [10]- | [6]- | [8]- | [10]- | |
|---|---|---|---|---|---|---|---|
| Sample | gingerol | gingerol | gingerol | shogaol | shogaol | shogaol | Total |
| Microwave- | — | — | — | — | — | — | — |
| processed | | | | | | | |
| ginger | | | | | | | |
| Processed | | | | | | | |
| product F1 | | | | | | | |
| MW ginger | — | — | — | — | — | — | — |
| Processed | | | | | | | |
| product F2 | | | | | | | |
| MW ginger | — | — | — | — | — | — | — |
| Processed | | | | | | | |
| product F3 | | | | | | | |
| MW ginger | 17.0 | 7.2 | — | 118.9 | — | — | 143.1 |
| Processed | | | | | | | |
| product F4 | | | | | | | |
| Microwave- | — | — | 3.9 | 41.9 | 24.3 | 36.6 | 106.7 |
| processed | | | | | | | |
| ginger | | | | | | | |
| Processed | | | | | | | |
| product F5 | | | | | | | |
| Microwave- | — | — | — | 0.5 | — | — | 0.5 |
| processed | | | | | | | |
| ginger | | | | | | | |
| Processed | | | | | | | |
| product F6 | | | | | | | |

As shown in Tables 3 and 4, INS-1 cells secreted insulin concentration-dependently in the presence of the ginger extract, the extract of ginger steamed for 6 hours, the extract of ginger steamed for 12 hours, the fraction of the ginger extract, the microwave-processed ginger product and a fraction thereof, and single compounds contained in ginger, such as [6]-gingerol, [8]-gingerol, [10]-gingerol, [6]-shogaol, [8]-shogaol, and [10]-shogaol. In particular, when comparing the ginger extract, the steamed ginger extracts, and the microwave-processed ginger product, the insulin secretion ability of the microwave-processed ginger product improved significantly. When comparing the fractions, the fraction of the processed ginger product in 80% ethanol (processed ginger product F4) was found to have the best insulin secretion ability. As a result of component analysis through UPLC, the highest activity was achieved when the content of [6]-shogaol, [8]-shogaol, and [10]-shogaol was 83% of the total gingerol and shogaol content in the fraction. In particular, when the content of [6]-shogaol was 80% or higher, high activity was achieved. Based on this result, from the results of Table 4 showing the insulin secretion ability of gingerols and shogaols, which are single components contained in ginger, [6]-shogaol was found to promote insulin secretion in INS-1 cells, as compared with other components.

(2) Results of Oral Glucose Tolerance Test (OGTT)

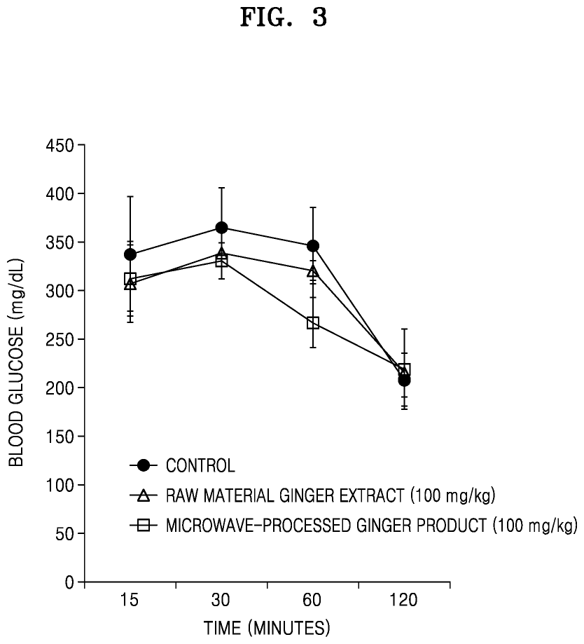

ICR mice were used for OGTT experiments. ICR mice were fasted for 12 hours before the experiment, blood was collected from the caudal vein of mice that had been fasted for 12 hours, and the fasting blood glucose level was measured as the initial blood glucose level. Thereafter, experiments were conducted with a group administered with glucose (2 g/Kg) (control), a group administered with glucose and a raw ginger extract (100 mg/kg, not processed), and a group administered with a microwave-processed ginger product (100 mg/kg, processed). After oral administration for each concentration, glucose was orally administered 1 hour later, and measurement was performed by using a blood glucose meter from the caudal vein at 0, 15, 30, 60, and 120 minutes. FIG. 3 is a graph of results of an oral glucose tolerance test of a ginger extract and a microwave-processed ginger product.

FIG. 3 shows results measured by oral glucose tolerance test by collecting blood from the caudal vein 15, 30, 60, and 120 minutes after oral administration of glucose to investigate the effect of the administration of ginger on oral glucose tolerance. As shown in FIG. 3, blood glucose started to increase 15 minutes after glucose administration. As compared with the control group and the group administered with glucose and the raw ginger extract, the increase in blood glucose level was significantly reduced in the group administered with glucose and the microwave-processed ginger product, and thus the microwave-processed ginger product was found to be effective in lowering glucose tolerance.

Next, to test the efficacy of a single component of ginger, blood was collected from the caudal vein of mice that had been fasted for 12 hours, and the fasting blood glucose level was measured as the initial blood glucose level. Thereafter, experiments were conducted with a group administered with glucose (2 g/Kg) (control), a group administered with glucose and 6-gingerol (100 mg/kg), and a group administered with glucose and 6-shogaol (100 mg/kg). After oral administration for each concentration of 6-gingerol and 6-shogaol, glucose was orally administered 1 hour later, and measurement was performed by using a blood glucose meter from the caudal vein at 0, 15, 30, 60, and 120 minutes.

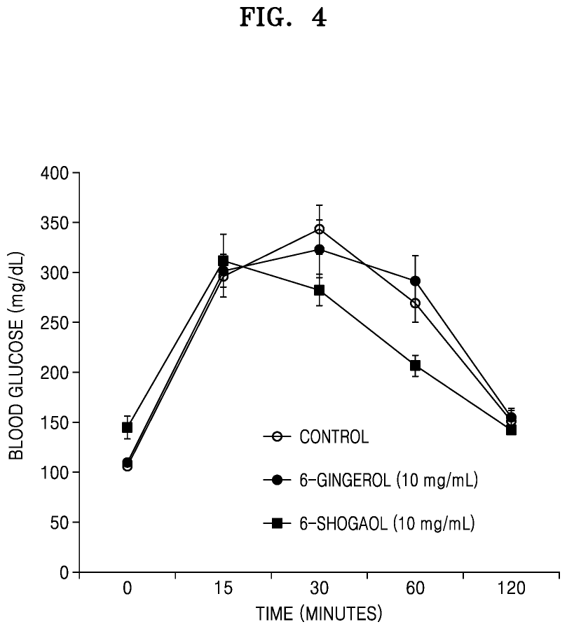

FIG. 4 is graph of results measured by oral glucose tolerance test by collecting blood from the caudal vein 15, 30, 60, and 120 minutes after oral administration of glucose to investigate the effect of administration of a single ingredient contained in ginger on oral glucose tolerance. As shown in FIG. 4, blood glucose started to increase 15 minutes after glucose administration. As compared with the control group and the group administered with glucose and 6-gingerol, the increase in blood glucose level was significantly reduced in the group administered with glucose and 6-shogaol, and thus 6-shogaol was found to be effective in lowering glucose tolerance.

From the above results, when a ginger extract was irradiated with microwaves, [6]-gingerol, [8]-gingerol, and [10]-gingerol were dehydrated to [6]-shogaol, [8]-shogaol, and [10]-shogaol, respectively, to thereby obtain a processed ginger product with an increased content of shogaol. In addition, it was found that the microwave-processed ginger products, in particular, the fraction (processed ginger product F4) with a high [6]-shogaol content and the [6]-shogaol single compound, stimulate the pancreatic cells to promote insulin secretion.

As apparent from the foregoing description, the antidiabetic composition according to an aspect may be used in preventing, improving, or treating diabetes in a subject.

The method of preventing or treating diabetes in a subject according to one aspect of the present disclosure may effectively prevent or treat diabetes.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A pharmaceutical composition for preventing or treating diabetes, the pharmaceutical composition comprising:
a ginger extract, as an active ingredient, obtained by microwave irradiation, under pressure, of ginger, an extract thereof, or a combination thereof, wherein in the ginger extract, a content of [6]-shogaol, [8]-shogaol, and [10]-shogaol is 20 wt % or more greater than a content of [6]-gingerol, [8]-gingerol, and [10]-gingerol, wherein the microwave irradiation is performed under a pressure in a range of about 2 atm to about 100 atm.

2. The pharmaceutical composition of claim 1, wherein the extract used in the microwave irradiation is extracted by using a solvent after the microwave irradiation.

3. The pharmaceutical composition of claim 1, wherein the microwave irradiation is performed at a temperature of about 120° C. to about 200° C. for about 30 minutes to about 90 minutes.

4. The pharmaceutical composition of claim 1, wherein the extract used in the microwave irradiation is a crude extract of ginger from water, a C₁-C₄ alcohol, or a mixture thereof; a solvent fraction of n-hexanet, methylene chloride, ethyl acetate, butanol, acetone, or a mixture thereof of the crude extrac; or a purified product of the solvent fraction.

5. The pharmaceutical composition of claim 1, wherein, in the ginger extract, a content of [6]-shogaol, [8]-shogaol, and [10]-shogaol is 80 wt % or greater of a total content of [6]-shogaol, [8]-shogaol, [10]-shogaol, [6]-gingerol, [8]-gingerol, and [10]-gingerol.

6. The pharmaceutical composition of claim 1, wherein, in the ginger extract, a content of [6]-shogaol is 80 wt % or greater of a total content of [6]-shogaol, [8]-shogaol, [10]-shogaol, [6]-gingerol, [8]-gingerol, and [10]-gingerol.

7. The pharmaceutical composition of claim 1, wherein, in the ginger extract, a content of [6]-shogaol, [8]-shogaol, and [10]-shogaol is 80 wt % or greater of a total content of [6]-shogaol, [8]-shogaol, [10]-shogaol, [6]-gingerol, [8]-gingerol, and [10]-gingerol.

8. The pharmaceutical composition of claim 1, wherein, in the ginger extract, a content of [6]-shogaol, [6]-gingerol, and [8]-gingerol is in a range of about 7.5 wt % to about 12.5 wt %; about 2.5 wt % to about 7.5 wt %; or about 80 wt % to about 90 wt %, based on a total content of [6]-shogaol, [8]-shogaol, [10]-shogaol, [6]-gingerol, [8]-gingerol, and [10]-gingerol.

9. The pharmaceutical composition of claim 1, wherein the ginger extract is a fraction obtained by fractionating a crude extract with ethanol.

10. The pharmaceutical composition of claim 9, wherein the ethanol is an about 70% to about 90% aqueous solution.

11. The pharmaceutical composition of claim 1, wherein the pharmaceutical composition promotes insulin secretion ability of cells.

12. A food composition for preventing or ameliorating diabetes, the pharmaceutical composition comprising:
a ginger extract obtained by microwave irradiation, under pressure, of ginger, an extract thereof, or a combination thereof, wherein in the ginger extract, a content of [6]-shogaol, [8]-shogaol, and [10]-shogaol is 20 wt % or more greater than a content of [6]-gingerol, [8]-gingerol, and [10]-gingerol, wherein the microwave irradiation is performed under a pressure in a range of about 2 atm to about 100 atm.

13. A method of preventing or treating diabetes in a subject, the method comprising administering the pharmaceutical composition to the subject.

14. The method of claim 13, wherein the extract used in the microwave irradiation is extracted by using a solvent after the microwave irradiation.

15. The method of claim 13, wherein the microwave irradiation is performed at a temperature of about 120° C. to about 200° C. for about 30 minutes to about 90 minutes.

16. The method of claim 13, wherein the extract used in the microwave irradiation is a crude extract of ginger from water, a C1-C4 alcohol, or a mixture thereof; a solvent fraction of n-hexanet, methylene chloride, ethyl acetate, butanol, acetone, or a mixture thereof of the crude extract or a purified product of the solvent fraction

17. The method of claim 13, wherein, in the ginger extract, a content of [6]-shogaol, [8]-shogaol, and [10]-shogaol is 80 wt % or greater of a total content of [6]-shogaol, [8]-shogaol, [10]-shogaol, [6]-gingerol, [8]-gingerol, and [10]-gingerol.

18. The method of claim 13, wherein, in the ginger extract, a content of [6]-shogaol is 80 wt % or greater of a total content of [6]-shogaol, [8]-shogaol, [10]-shogaol, [6]-gingerol, [8]-gingerol, and [10]-gingerol.

19. The method of claim 13, wherein, in the ginger extract, a content of [6]-shogaol, [8]-shogaol, and [10]-shogaol is 80 wt % or greater of a total content of [6]-shogaol, [8]-shogaol, [10]-shogaol, [6]-gingerol, [8]-gingerol, and [10]-gingerol.

20. The method of claim 13, wherein, in the ginger extract, a content of [6]-shogaol, [6]-gingerol, and [8]-gingerol is in a range of about 7.5 wt % to about 12.5 wt %; about 2.5 wt % to about 7.5 wt %; or about 80 wt % to about 90 wt %, based on a total content of [6]-shogaol, [8]-shogaol, [10]-shogaol, [6]-gingerol, [8]-gingerol, and [10]-gingerol.
